(19) United States
(12) Patent Application Publication
(10) Pub. No.: US 2006/0064108 A1
(43) Pub. Date: Mar. 23, 2006

(54) Tibial sizing apparatus and method

(76) Inventors: Jeffrey C. Blaylock, Fort Wayne, IN (US); John E. Meyers, Columbia City, IN (US); John E. Pendleton, Fort Wayne, IN (US); Susan W. Zogbi, Winona Lake, IN (US)

Correspondence Address: ZIMMER TECHNOLOGY - ROBERTS, P.O. BOX 1268, ALEDO, TX, 76008, US

(21) Appl. No.: 10/938,979

(22) Filed: Sep. 9, 2004

(51) Int. Cl. A61B 17/60

(52) U.S. Cl. 606/102

(57) ABSTRACT

A tibial sizing apparatus includes a tibial sizing plate. The tibial sizing plate includes a substantially planar surface and defines a peripherally positioned pin slot extending obliquely relative to the substantially planar surface. An alternative tibial sizing apparatus includes a tibial sizing plate and an angulated handle coupled to the tibial sizing plate. An alternative tibial sizing apparatus includes a tibial sizing plate and a catch member coupled to a peripheral portion of the tibial sizing plate. The catch member is movable between a first position in which the catch member extends generally inferiorly from the tibial sizing plate and a second position in which the catch member retracts generally superiorly into the tibial sizing plate. A method for fixing a tibial sizing plate to a proximal tibia includes anteriorly pinning the tibial sizing plate to the proximal tibia.

### FIELD OF THE INVENTION

The present invention relates generally to the field of orthopaedics, and, more particularly, to a tibial sizing apparatus and a method for fixing a tibial sizing plate to a proximal tibia.

### BACKGROUND

Total joint arthroplasty (“joint replacement”) is the surgical replacement of a joint with a prosthesis. A typical knee prosthesis has three main components: a femoral implant, a tibial implant, and a tibio-femoral insert. In general, the femoral implant is designed to replace the distal femoral condyles. The femoral implant is typically made from metal. It typically includes a head portion (“femoral head”) having rounded surfaces for emulating the condyles, and an elongated stem extending away from the femoral head for anchoring the femoral implant in the intramedullary canal of the distal femur. In general, the tibial implant is designed to support and align the tibio-femoral insert. The tibial implant is also typically made from metal. It typically includes a substantially planar tray or plate portion (“tibial plate”) for supporting the insert, and an elongated stem extending away from the tibial plate for anchoring the tibial implant in the intramedullary canal of the proximal tibia. In general, the tibio-femoral insert is designed to replace the tibial plateau and the meniscus of the knee. It is typically somewhat disk-shaped, and typically includes one or more substantially planar surfaces for bearing on the tibial plate and one or more generally concave surfaces for bearing against the femoral head. The insert is typically made of a strong, smooth, low-wearing plastic.

In a traditional knee replacement, the surgeon makes a rather lengthy anterior incision spanning over the distal femur, the knee, and the proximal tibia; everts the patella; separates the distal femur and proximal tibia from the surrounding tissues; hyperflexes, distally extends, and/or otherwise moves the proximal tibia away from the distal femur to make room for specialized jigs, saws, drills, and/or other instruments; and uses the instruments to prepare the distal femur and the proximal tibia for receiving the prosthetics. Finally, the surgeon drives the stems of the femoral implant and tibial implant generally longitudinally into the intramedullary canals of the distal femur and proximal tibia, respectively; cements each stem in place; aligns and/or attaches the insert onto the tibial plate; aligns and/or attaches the femoral head onto the insert; reverts the patella; and closes the surgical site.

In contrast to a traditional knee replacement, knee replacement through minimally invasive surgery employs, among other things, smaller incisions, which tend to reduce tissue traumas and accelerate post-operative recoveries. Minimally invasive surgical techniques are becoming increasingly popular. However, because minimally invasive techniques generally reduce the size of the surgical site, they also generally reduce the amount of space available for inserting and manipulating arthroplasty instruments. Moreover, in some minimally invasive knee replacements the patella is never everted. In such procedures, the need to work around the patella can present additional challenges.

A tibial sizing plate is a specialized jig used in knee replacement operations. The typical tibial sizing plate provides one or more apertures and/or surfaces for aligning one or more of a broach, a drill, and/or other finishing tools for further preparation of a resected proximal tibia to receive a tibial implant. Tibial sizing plates are typically available in various sizes. During the knee replacement, a particular size is selected according to the general size of the resected proximal tibia and corresponding design configurations and interplays between the various parts of the ultimate prosthesis. The typical tibial sizing plate is configured to be aligned on the resected proximal tibia relative to anatomic landmarks and/or by trial flexions and extensions of provisional parts which may include a provisional femoral component and/or a provisional tibio-femoral component. The typical tibial sizing plate is also configured to be temporarily pinned or screwed in place on the resected proximal tibia after alignment.

Historical tibial sizing plates have been designed primarily for use in traditional knee replacement procedures, where space around the surgical site has generally been more plentiful and access to the surgical site has generally been more direct than in minimally invasive surgeries.

### SUMMARY OF THE INVENTION

The present invention provides a tibial sizing apparatus including a tibial sizing plate. The tibial sizing plate includes a substantially planar surface and defines a peripherally positioned pin slot extending obliquely relative to the substantially planar surface.

The present invention provides a tibial sizing apparatus including a tibial sizing plate and an angulated handle coupled to the tibial sizing plate.

The present invention provides a tibial sizing apparatus including a tibial sizing plate and a first catch member coupled to a first peripheral portion of the tibial sizing plate. The first catch member is movable between a first first catch member position in which the first catch member extends generally inferiorly from the tibial sizing plate and a second first catch member position in which the first catch member retracts generally superiorly into the tibial sizing plate.

The present invention provides a method for fixing a tibial sizing plate to a proximal tibia. The method includes anteriorly pinning the tibial sizing plate to the proximal tibia.

### BRIEF DESCRIPTION OF THE DRAWINGS

FIG. 1 shows a perspective view of an exemplary tibial sizing apparatus according to the present invention;

FIG. 2 shows a superior plan view of the exemplary tibial sizing plate of the exemplary tibial sizing apparatus of FIG. 1;

FIG. 3 shows an inferior plan view of the exemplary tibial sizing plate of the exemplary tibial sizing apparatus of FIG. 1;

FIG. 4 shows a perspective view of the exemplary tibial sizing plate of the exemplary tibial sizing apparatus of FIG. 1;

FIG. 5 shows a plan view of the exemplary retractable posterior alignment post of the exemplary tibial sizing apparatus of FIG. 1;

FIG. 6 shows a plan view of an exemplary alternative retractable posterior alignment post;

FIG. 7 shows a superior plan view of the exemplary tibial sizing apparatus of FIG. 1;

FIG. 8 shows a medial plan view of the exemplary tibial sizing apparatus of FIG. 1;

FIG. 9 shows a cross-sectional view of the exemplary angulated handle of the exemplary tibial sizing apparatus of FIG. 1 taken along line 9-9 of FIG. 8;

FIG. 10 shows a perspective view of the exemplary alignment rod of the exemplary tibial sizing apparatus of FIG. 1;

FIG. 11 shows a medial plan view of exemplary operations of the exemplary tibial sizing apparatus of FIG. 1 during a left knee replacement in which a patella is not everted; and

FIG. 12 shows a superior plan view of an exemplary alternative tibial sizing plate according to the present invention.

### DETAILED DESCRIPTION OF EXEMPLARY EMBODIMENT(S)

As used herein, the terms “medial,” “medially,” and the like mean pertaining to the middle, in or towards the middle, and/or nearer to the middle of the body when standing or positioned upright. Conversely, the terms “lateral,” “laterally,” and the like are used herein as opposed to medial and/or corresponding terms. For example, the medial side of the knee is the side closest to the other knee and the closest sides of the knees are medially facing, whereas the lateral side of the knee is the outside of the knee and is laterally facing. Further, as used herein the terms “superior,” “superiorly,” and the like mean closer to the top of the head and/or farther from the bottom of the feet when standing or positioned upright. Conversely, the terms “inferior,” “inferiorly,” and the like are used herein as opposed to superior and/or corresponding terms. For example, the heart is superior to the stomach and the superior surface of the tongue rests against the palate, whereas the stomach is inferior to the heart and the palate faces inferiorly towards the tongue. Additionally, as used herein the terms “anterior,” “anteriorly,” and the like mean nearer the front or facing away from the front of the body when standing or positioned upright, as opposed to “posterior,” “posteriorly,” and the like, which mean nearer the back or facing away from the back of the body when standing or positioned upright. Like reference numerals refer to like parts throughout the following description and the accompanying drawings.

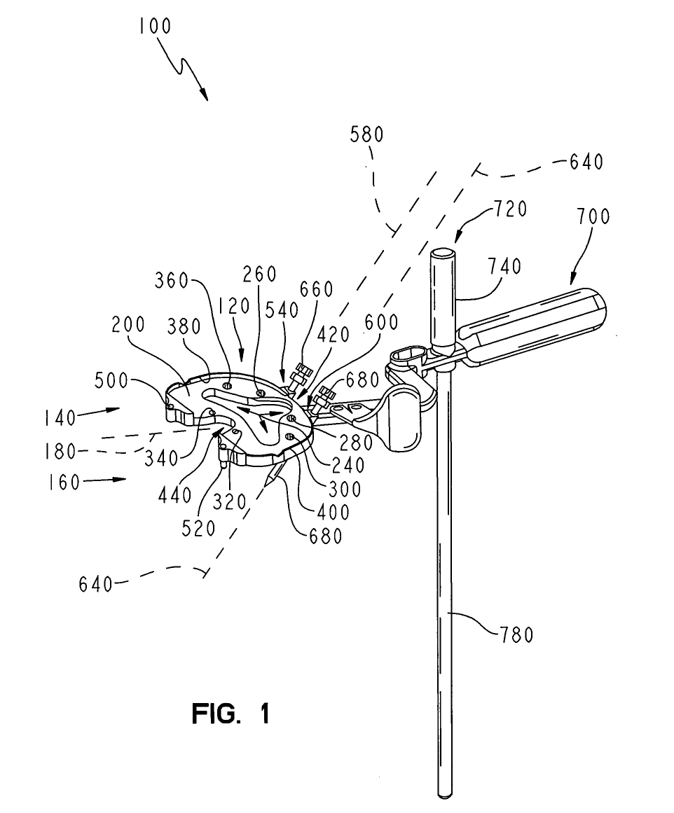

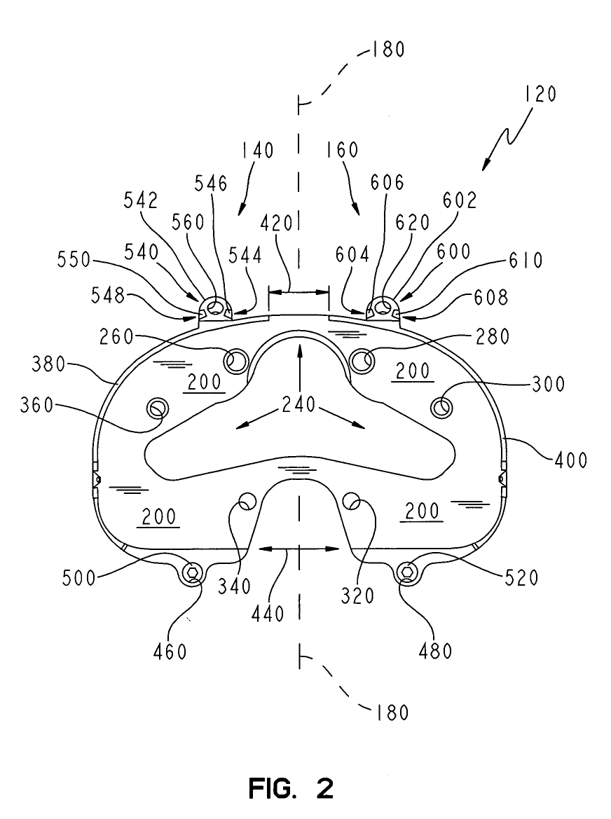

FIG. 1 shows a perspective view of an exemplary tibial sizing apparatus 100 according to the present invention. Apparatus 100 includes an exemplary tibial sizing plate 120. Plate 120 may be thought of as having a side or lobe 140 and an opposing side or lobe 160 relative to an imaginary medial-lateral split line or center line 180 (see also FIG. 2). Plate 120 is configured to, among other things, provide one or more apertures and/or surfaces for aligning one or more of a broach, a drill, and/or other finishing tools (not shown) for further preparation of a resected proximal tibia (not shown) to receive a tibial implant (not shown). In the exemplary embodiment, plate 120 is made from titanium. Among other things, the magnetic resonance properties of titanium facilitate compatibility with surgical imaging and guidance systems. In alternative embodiments, plate 120 may be made from stainless steel or any other suitable material.

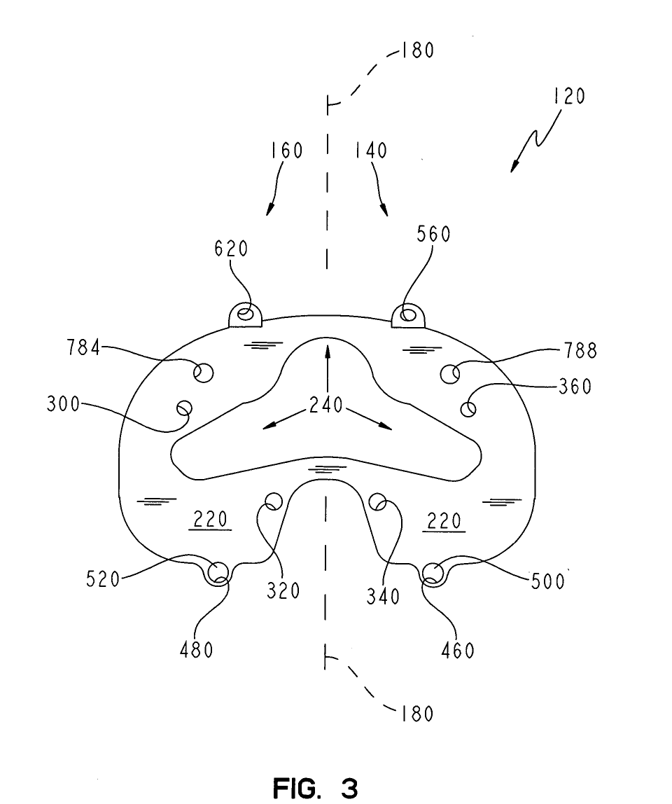

Plate 120 includes a substantially planar superior surface 200 (see also FIG. 2) and a substantially planar inferior surface 220 (see FIG. 3). Plate 120 defines a somewhat rounded, generally triangular, slightly V-shaped aperture 240 extending perpendicularly between surface 200 and surface 220 (see also FIG. 2 and FIG. 3). Plate 120 also defines a substantially cylindrical indentation 260 and a substantially cylindrical indentation 280 extending inferiorly from surface 200 (see also FIG. 2). Plate 120 also defines a cylindrical aperture 300, a cylindrical aperture 320, a cylindrical aperture 340, and a cylindrical aperture 360 extending perpendicularly between surface 200 and surface 220 (see also FIG. 2 and FIG. 3). It should be appreciated that aperture 240, indentation 260, indentation 280, aperture 300, aperture 320, aperture 340, and aperture 360 may facilitate alignment of one or more of a broach, a drill, and/or other finishing tools for further preparation of a resected proximal tibia to receive a tibial implant (not shown).

Lobe 140 of plate 120 includes a peripheral wall 380 extending generally perpendicularly away from surface 200, while lobe 160 of plate 120 includes a peripheral wall 400 extending generally perpendicularly away from surface 200 (see also FIG. 2). Wall 380 and wall 400 do not meet anteriorly or posteriorly on plate 120. Instead, wall 380 and wall 400 are spaced apart to define an anterior notch 420 and a posterior notch 440 (see also FIG. 2). It should be appreciated that wall 380, wall 400, notch 420, and notch 440 may facilitate alignment and/or retention of a provisional tibio-femoral insert (not shown).

Wall 380 protrudes posteriorly in a rounded or curved fashion to define a posteriorly positioned interiorly screw-threaded generally cylindrical aperture 460 which extends perpendicularly to surface 200 (see FIG. 2 and FIG. 3). Wall 400 protrudes posteriorly in a rounded or curved fashion to define a posteriorly positioned interiorly screw-threaded generally cylindrical aperture 480 which extends perpendicularly to surface 200 (see FIG. 2 and FIG. 3).

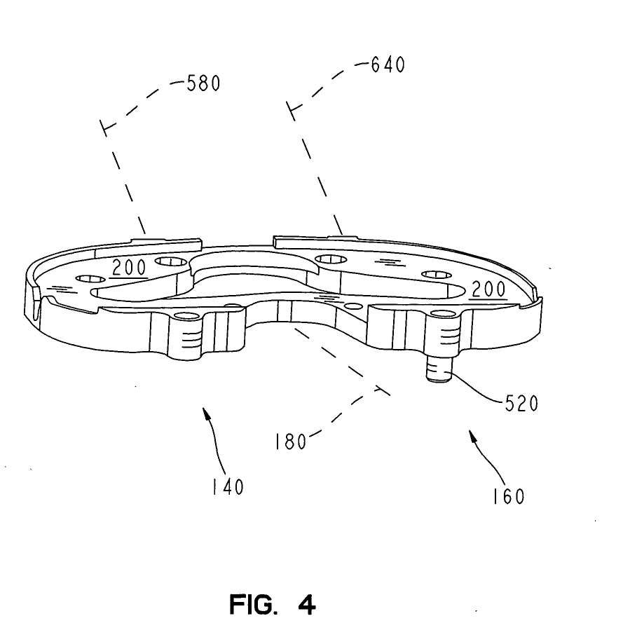

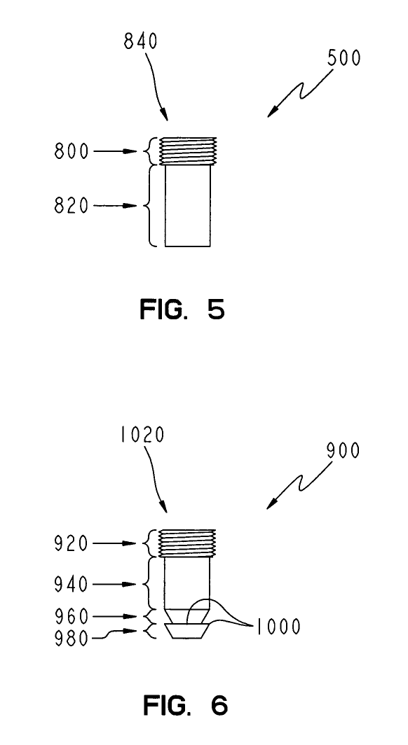

Plate 120 further includes a post 500 extendably and retractably screwed into aperture 460 and an identical post 520 extendably and retractably screwed into aperture 480 (see also FIG. 2, FIG. 3, FIG. 4, and FIG. 5). It should be appreciated that in the pertinent exemplary drawings, post 500 is shown retracted into aperture 460 while post 520 is shown extending from aperture 480. Post 500 and post 520 are discussed further below.

Plate 120 further includes a notched protuberance 540 extending generally anteriorly from wall 380. Protuberance 540 includes a rounded or curved anterior surface 542. Further, protuberance 540 defines a medial notch 544 bounded inferiorly by a substantially planar surface 546 and a lateral notch 548 bounded inferiorly by a substantially planar surface 550 (see FIG. 2). Protuberance 540 also defines a substantially cylindrical pin slot 560 (see FIG. 2). Slot 560 extends through protuberance 540—obliquely relative to surface 200—about an axis 580 (see also FIG. 4). In other words, axis 580 is neither perpendicular nor parallel to surface 200.

Plate 120 further includes a notched protuberance 600 extending generally anteriorly from wall 400. Protuberance 600 includes a rounded or curved anterior surface 602. Further, protuberance 600 defines a medial notch 604 bounded inferiorly by a substantially planar surface 606 and a lateral notch 608 bounded inferiorly by a substantially planar surface 610 (see FIG. 2). Protuberance 600 also defines a substantially cylindrical pin slot 620 (see FIG. 2). Slot 620 extends through protuberance 600—obliquely relative to surface 200—about an axis 640 (see also FIG. 4). In other words, axis 640 is neither perpendicular nor parallel to surface 200. In the exemplary embodiment, axis 640 is parallel to axis 580.

Apparatus 100 further includes a bone pin 660 extending through slot 560 and an identical bone pin 680 extending through slot 620. Pin 660 and pin 680 are each configured to be hammered or otherwise driven into the proximal tibia to secure plate 120 thereto. Pin 660 and pin 680 are each made of stainless steel or any other suitable material. In alternative embodiments, each of pin 660 and/or pin 680 may be replaced with a suitable bone screw or any other suitable fastener.

Apparatus 100 further includes a handle 700 removably coupled to one of protuberance 540 and protuberance 600. It should be appreciated that in the pertinent exemplary drawings, handle 700 is shown removably coupled to protuberance 600. Handle 700 is configured to, among other things, facilitate manipulation and/or alignment of plate 120. Handle 700 is discussed further below.

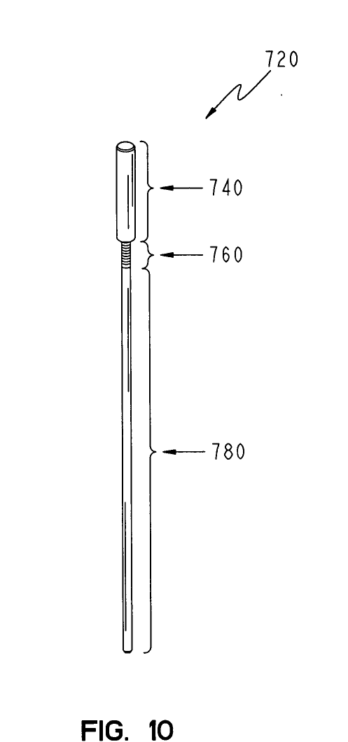

Apparatus 100 further includes an alignment rod 720. Rod 720 is configured to, among other things, facilitate alignment of plate 120 during operation of apparatus 100. In the exemplary embodiment, rod 720 is made from 17-4 stainless steel. In alternative embodiments, rod 720 may be made from any other suitable material. Rod 720 includes a substantially cylindrical portion 740, an exteriorly screw-threaded generally cylindrical portion 760 (see FIG. 10) extending inferiorly from portion 740 through handle 700 as discussed further below, and a substantially cylindrical non-threaded portion 780 extending inferiorly from portion 760. Rod 720 is discussed further below.

FIG. 2 shows a superior plan view of plate 120. Lobe 140, lobe 160, line 180, surface 200, aperture 240, indentation 260, indentation 280, aperture 300, aperture 320, aperture, 340, aperture 360, wall 380, wall 400, notch 420, notch 440, aperture, 460, aperture 480, post 500, post 520, protuberance 540, notch 544, notch 548, slot 560, protuberance 600, notch 604, notch 608, and slot 620, among other things, are all at least partially discernable in FIG. 2.

FIG. 3 shows an inferior plan view of plate 120. As discernable in FIG. 3, plate 120 also defines a cylindrical indentation 784 and a cylindrical indentation 788 extending superiorly from surface 220. It should be appreciated that indentation 784 and indentation 788 may facilitate alignment and/or attachment of a spacer or other augmentation (not shown) inferiorly to plate 120. Lobe 140, lobe 160, line 180, surface 220, aperture 240, aperture 300, aperture 320, aperture 340, aperture 360, aperture 460, aperture 480, post 500, post 520, slot 560, and slot 620, among other things, are all at least partially discernable in FIG. 3.

FIG. 4 shows a perspective view of plate 120. Lobe 14, lobe 160, line 180, post 520, axis 580, and axis 640, among other things, are all at least partially discernable in FIG. 4.

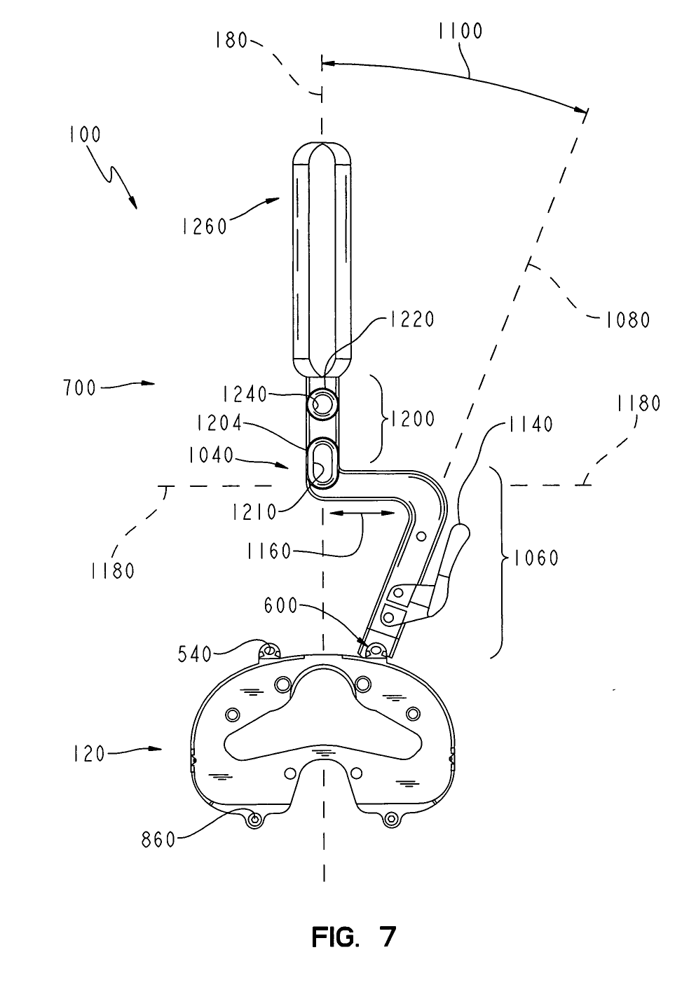

FIG. 5 shows a plan view of post 500. Post 500 is configured to, among other things, be extendably and retractably screwed into aperture 460 of plate 120. Post 500 is made from stainless steel or any other suitable material, and includes an exteriorly screw-threaded generally cylindrical head portion 800 and a non-threaded cylindrical body portion 820 extending inferiorly from portion 800. Portion 800 includes a superior surface 840. In the exemplary embodiment, surface 840 defines a hex socket 860 (see, e.g., FIG. 7). Socket 860 is suitable for receiving the tip of a hex head screw driver or the like. In alternative embodiments, surface 840 may define any other suitable slot or socket for receiving a flat head screwdriver or any other suitable instrument. As stated above, post 520 is configured identically to post 500.

FIG. 6 shows a plan view of an exemplary alternative retractable posterior alignment post 900. In alternative embodiments, post 900 replaces post 500 and an identically configured post replaces post 520. Post 900 is configured to, among other things, be extendably and retractably screwed into aperture 460 of plate 120. Post 900 is made from stainless steel or any other suitable material, and includes an exteriorly screw-threaded generally cylindrical head portion 920, a non-threaded cylindrical body portion 940 extending inferiorly from portion 920, a non-threaded frusto-conical portion 960 extending inferiorly from portion 940, and a non-threaded frusto-conical portion 980 extending inferiorly from portion 960. Portion 980 includes an annular edge 1000. Portion 920 includes a superior surface 1020. Surface 1020 defines a slot (not discernable in FIG. 6) identically configured to slot 860 of post 500 (discussed above).

FIG. 7 shows a superior plan view of apparatus 100. Plate 120 (including protuberance 540, protuberance 600 and slot 860) and handle 700, among other things, are all at least partially discernable in FIG. 7. As stated above, handle 700 is configured to, among other things, facilitate manipulation and/or alignment of plate 120.

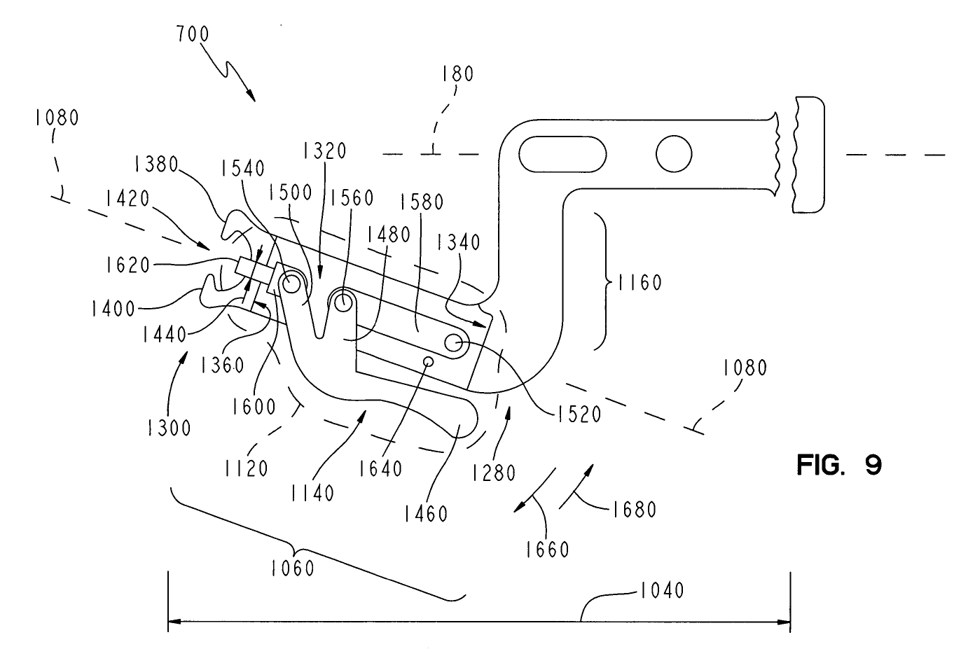

Handle 700 includes an angulated arm 1040 made from stainless steel or any other suitable material. Arm 1040 includes a longitudinal portion 1060. Portion 1060 is configured to, among other things, removably couple to either one of protuberance 540 and protuberance 600 as discussed further below and to extend somewhat anteriorly therefrom and away from line 180 along a line 1080 at an angle 1100 relative to line 180. In FIG. 7, portion 1060 is shown removably coupled to protuberance 600. In the exemplary embodiment, angle 1100 is preferably 20 degrees, which facilitates bypassing or working around a patella and a patellar tendon during procedures in which the patella is not everted. In alternative embodiments, angle 1100 may be any other suitable number of degrees. Portion 1060 houses an interference coupling mechanism 1120 (see FIG. 9) which includes a branched actuation lever 1140. Mechanism 1120 is configured to cooperate with portion 1060 to removably couple arm 1040 to either one of protuberance 540 and protuberance 600. Portion 1060 and mechanism 1120 are discussed further below.

Arm 1040 also includes a longitudinal portion 1160 extending from portion 1060 to line 180 along a line 1180 that is perpendicular to line 180. Further, arm 1040 includes a longitudinal portion 1200 extending anteriorly from portion 1160 along line 180. It should be appreciated, then, that portion 1200 extends perpendicularly away from portion 1160 along line 180. Portion 1160 and portion 1200 together define an elongated superiorly extending collar 1204. Portion 1160, portion 1200, and collar 1204 define a superior-inferior smooth, non-threaded elongated aperture 1210 oriented longitudinally along line 180. Arm 1040 also includes a cylindrical collar 1220 extending superiorly from portion 1200. Portion 1200 and collar 1220 define a superior-inferior smooth, non-threaded cylindrical aperture 1240. In alternative embodiments, aperture 1240 may be internally screw-threaded to mate with portion 760 of rod 720 as discussed further below.

Handle 700 also includes a longitudinal grip 1260 extending anteriorly from portion 1200 of arm 1040 along line 180. Grip 1260 is configured to be grasped by hand, and is made from stainless steel or any other suitable material.

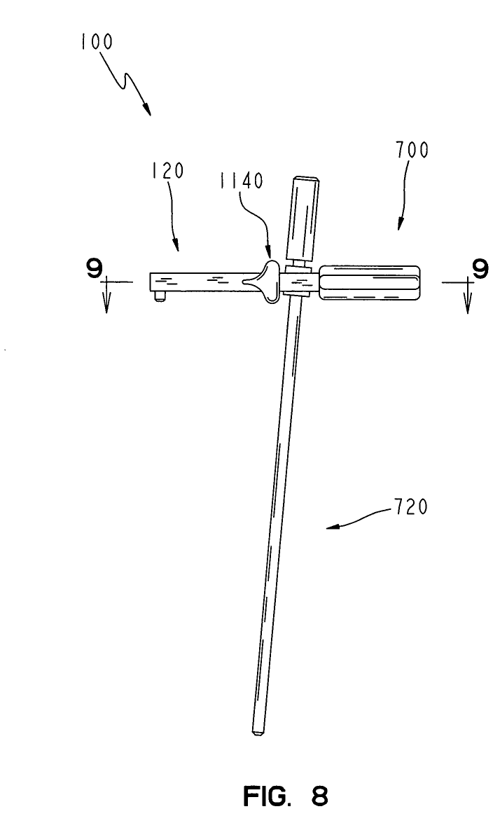

FIG. 8 shows a medial plan view of apparatus 100. Plate 120, handle 700, rod 720, and lever 1140, among other things, are all at least partially discernable in FIG. 8.

FIG. 9 shows a cross-sectional view of handle 700 taken along line 9-9 of FIG. 8. As stated above, portion 1060 of arm 1040 houses mechanism 1120, which is discussed further below. To accommodate and cooperate with mechanism 1120, among other things, portion 1060 includes an end 1280 merging with and extending from portion 1160 of arm 1040 and further includes an opposing end 1300. Between end 1280 and end 1300, portion 1060 defines a longitudinal substantially rectilinear cavity 1320 bounded proximally to end 1280 by a wall 1340 and bounded proximally to end 1300 by a wall 1360. Further, end 1300 defines a prong 1380 spaced apart from yet pointing toward line 1080, an opposing identically configured prong 1400 spaced apart from yet pointing toward line 1080, a generally hemi-cylindrical space 1420 extending between prong 1380 and prong 1400, and a cylindrical aperture 1440 extending axially along line 1080 through wall 1360 (equidistantly between prong 1380 and prong 1400).

Mechanism 1120 is configured to cooperate with portion 1060 of arm 1040 to removably couple arm 1040 to either one of protuberance 540 (see FIG. 1, FIG. 2, and FIG. 7) and protuberance 600 (see FIG. 1, FIG. 2, and FIG. 7). As noted above, mechanism 1120 includes lever 1140. Lever 1140 is configured to, among other things, be actuated by one or more fingers and/or a thumb and includes a boom 1460 positioned outside of cavity 1320, a branch 1480 forking from boom 1460 into cavity 1320, and a branch 1500 forking from boom 1460 and branch 1480 into cavity 1320. Mechanism 1120 also includes a hinge pin 1520 positioned proximally to wall 1340 along line 1080 within cavity 1320, a hinge pin 1540 positioned proximally to wall 1360 along line 1080 within cavity 1320, a hinge pin 1560 positioned between pin 1520 and pin 1540 within cavity 1320, a link 1580 pivotally coupled to pin 1520 and pivotally coupled to pin 1560, a link 1600 pivotally coupled to pin 1540, a post 1620 extending axially along line 1080 from link 1600 through aperture 1440, and a stop pin 1640 fixedly positioned within cavity 1320 generally between link 1580 and boom 1460.

To removably couple arm 1040 (and, thus, handle 700) to protuberance 600 (see also FIG. 1 and FIG. 7), boom 1460 is pivoted away from end 1280 of portion 1060 of arm 1040 as indicated generally by clockwise directional line 1660, which in turn retracts post 1620 from cavity 1420. Pin 1640 prevents excessive clockwise pivoting of boom 1460 by limiting the corresponding pivoting of link 1580, which in turn prevents post 1620 from sliding all the way out of aperture 1440. Next, arm 1040 is positioned relative to plate 120 such that prong 1380 is superior to notch 604 and prong 1400 is superior to notch 608. Arm 1040 is then repositioned such that prong 1380 slides inferiorly into notch 604 until it stops against surface 606 and prong 1400 slides inferiorly into notch 608 until it stops against surface 610 (and, thus, protuberance 600 extends into cavity 1420). Boom 1460 is then pivoted back towards end 1280 as indicated generally by counterclockwise directional line 1680, which in turn urges post 1620 back towards cavity 1420. When boom 1460 is pivoted back towards end 1280, the forking of branch 1480 and branch 1500 provides a spring force along line 1080 that clamps protuberance 600 between post 1620, prong 1380, and prong 1400.

To removably couple arm 1040 (and, thus, handle 700) to protuberance 540 (see also FIG. 1 and FIG. 7), boom 1460 is pivoted away from end 1280 of portion 1060 of arm 1040 as indicated generally by clockwise directional line 1660, which in turn retracts post 1620 from cavity 1420. Pin 1640 prevents excessive clockwise pivoting of boom 1460 by limiting the corresponding pivoting of link 1580, which in turn prevents post 1620 from sliding all the way out of aperture 1440. Next, handle 700 is decoupled from protuberance 600 and rotated 180 degrees about line 180 (i.e., turned over or upside down). Arm 1040 is then positioned relative to plate 120 such that prong 1380 is superior to notch 544 and prong 1400 is superior to notch 548. Arm 1040 is then repositioned such that prong 1380 slides inferiorly into notch 544 until it stops against surface 546 and prong 1400 slides inferiorly into notch 548 until it stops against surface 550 (and, thus, protuberance 540 extends into cavity 1420 as well). Boom 1460 is then pivoted back towards end 1280 as indicated generally by counterclockwise directional line 1680, which in turn urges post 1620 back towards cavity 1420. When boom 1460 is pivoted back towards end 1280, the forking of branch 1480 and branch 1500 provides a spring force along line 1080 that clamps protuberance 540 between post 1620, prong 1380, and prong 1400.

FIG. 10 shows a perspective view of rod 720. In the exemplary embodiment, portion 760 of rod 720 extends through aperture 1240 of arm 1040 of handle 700, while portion 780 extends generally inferiorly from aperture 1240 and portion 740 stops on collar 1220 and extends generally superiorly from aperture 1240 (see FIG. 1 and FIG. 7). In the exemplary embodiment, portion 760 merely slides into aperture 1240 (as aperture 1240 is not threaded). In alternative embodiments in which aperture 1240 is internally screw-threaded, portion 760 is screwed into aperture 1240 to removably couple rod 720 to handle 700 (see also FIG. 7). In other alternative embodiments, rod 720 is inserted through handle 700 such that portion 760 extends through aperture 1210 rather than aperture 1240 (see also FIG. 7). It should be appreciated that such embodiments provide some flexibility to slide rod 720 anteriorly or posteriorly along line 180 on collar 1204.

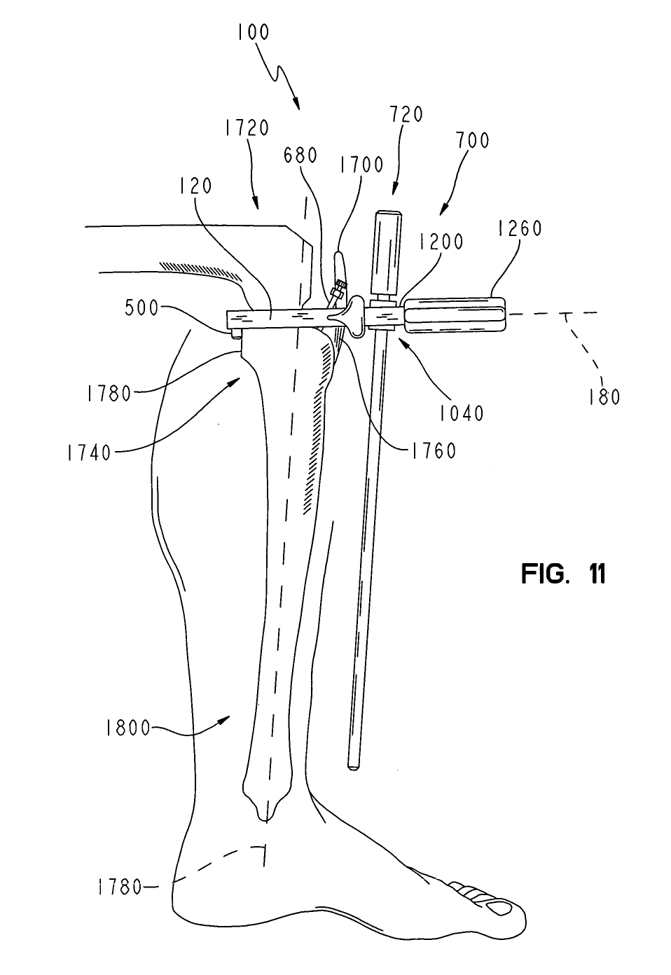

FIG. 11 shows a medial plan view of exemplary operations of apparatus 100 during a left knee replacement in which a patella 1700 is not everted. As shown in FIG. 11, a left distal femur 1720 and a left proximal tibia 1740 have already been resected (at least roughly). Removably coupling handle 700 to one of protuberance 540 and protuberance 600 is discussed above. If more lateral access to the left knee is desired than medial access, handle 700 is removably coupled to protuberance 600; whereas, if more medial access to the knee is desired than lateral access, arm 1040 of handle 700 is removably coupled to protuberance 540. In FIG. 11, handle 700 is shown coupled to protuberance 600.

If lateral posterior alignment of plate 120 is desired for the left knee replacement, a screwdriver is applied to post 500 to extend post 500 from aperture 460 and the screwdriver is applied post 520 to retract post 520 into aperture 480; whereas, if medial posterior alignment of plate 120 is desired for the left knee replacement, a screwdriver is applied to post 500 to retract post 500 into aperture 460 and the screwdriver is applied post 520 to extend post 520 from aperture 480. In FIG. 11, post 500 is shown extended and post 520 is shown retracted for lateral posterior alignment of plate 120 during the left knee replacement.

Next, handle 700 is manipulated to facilitate alignment of plate 120 on proximal tibia 1740. Post 500 (or post 520 as the case may be) facilitates alignment of plate 120 by catching or stopping against a corresponding medial or lateral posterior surface or anatomic reference 1780 on proximal tibia 1720. The rounding of wall 380 around slot 460 (which houses post 500), the rounding of wall 400 around slot 480 (which houses post 520), rounded surface 542 of protuberance 540, and rounded surface 602 of protuberance 600 facilitate avoidance of snagging in the surrounding soft tissues. Meanwhile, the angularity of handle 700 provides anterior clearance for working around patella 1700 and around a corresponding patellar tendon 1760 that extends between patella 1700 and proximal tibia 1740.

After plate 120 is aligned on proximal tibia 1740 as desired, it is secured to proximal tibia 1740 by hammering pin 660 into proximal tibia 1740 (through slot 560) and/or by hammering pin 680 into proximal tibia 1740 (through slot 620). The obliqueness of slot 560 and slot 620 relative to surface 220 allows pin 660 and/or pin 680 to be installed in a generally anterior fashion—as opposed to the substantially superior pinning required by traditional tibial sizing plates. In general, superior pinning requires more working space than the anterior pinning provisions of the present invention. Moreover, the anterior pinning allows fixation of plate 120 to proximal tibia 1740 with a tibio-femoral insert (not shown) positioned and/or retained on plate 120—without removal of the tibio-femoral insert as required by traditional tibial sizing plates. Additionally, the extension of portion 1200 of arm 1040 and grip 1260 along line 180 facilitates a determination of a provisional rotation angle for plate 120, and rod 720 facilitates comparisons of the position of plate 120 to the long axis 1780 of the left tibia 1800. In any event, after plate 120 is pinned to proximal tibia 1740, aperture 240, indentation 260, indentation 280, aperture 300, aperture 320, aperture 340, and aperture 360 facilitate alignment of one or more of a broach, a drill, and/or other finishing tools for further preparation of proximal tibia 1740 to receive a tibial implant (not shown).

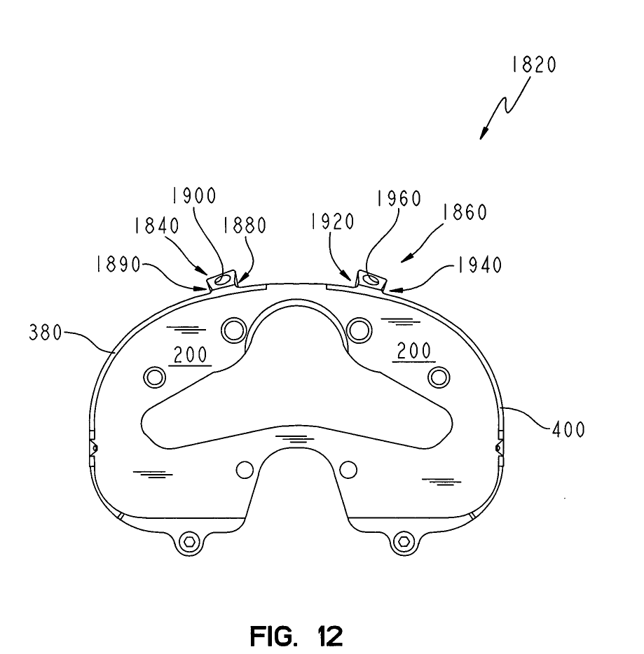

FIG. 12 shows a superior plan view of an exemplary alternative tibial sizing plate 1820 according to the present invention. Exemplary plate 1820 is configured identically to exemplary plate 120 except for flared protuberance 1840 (extending generally anteriorly from wall 380) and flared protuberance 1860 (extending generally anteriorly from wall 400), which replace protuberance 540 and protuberance 600, respectively. Protuberance 1840 and wall 380 together form a medial notch 1880 and a lateral notch 1890 about protuberance 1840. Protuberance 1840 further defines a substantially cylindrical pin slot 1900. Slot 1900 extends through protuberance 1840 obliquely relative to surface 200. Similarly, protuberance 1860 and wall 400 together form a medial notch 1920 and a lateral notch 1940 about protuberance 1860. Protuberance 1860 further defines a substantially cylindrical pin slot 1960. Slot 1960 extends through protuberance 1860 obliquely relative to surface 200. Plate 1820 is used similarly to plate 120.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. A tibial sizing apparatus, comprising:
a tibial sizing plate including a substantially planar surface and defining a peripherally positioned pin slot extending obliquely relative to the substantially planar surface.

2. The apparatus of claim 1, wherein the tibial sizing plate includes titanium.

3. The apparatus of claim 1, wherein the tibial sizing plate is configured such that when the substantially planar surface faces inferiorly the pin slot is additionally positioned generally anteriorly of the substantially planar surface.

4. The apparatus of claim 3, further comprising at least one of a bone pin and a bone screw extending through the pin slot.

5. The apparatus of claim 3, wherein the tibial sizing plate further includes a flared portion and at least a portion of the pin slot extends through the flared portion.

6. The apparatus of claim 3, wherein the tibial sizing plate further includes a notched portion and at least a portion of the pin slot extends through the notched portion.

7. The apparatus of claim 6, wherein the notched portion includes a substantially planar surface and the notched portion defines a notch bounded inferiorly by the substantially planar surface of the notched portion.

8. The apparatus of claim 3, further comprising:
a catch member coupled to a peripheral portion of the tibial sizing plate;
wherein the catch member is movable between a first position in which the catch member extends generally inferiorly from the tibial sizing plate and a second position in which the catch member retracts generally superiorly into the tibial sizing plate.

9. The apparatus of claim 8, wherein the catch member is positioned generally posteriorly of the substantially planar surface of the tibial sizing plate.

10. The apparatus of claim 9, wherein the catch member includes a pin.

11. The apparatus of claim 10, wherein the pin includes an annular notch.

12. A tibial sizing apparatus, comprising:
a tibial sizing plate; and
an angulated handle coupled to the tibial sizing plate.

13. The apparatus of claim 12, wherein the handle includes a substantially straight first portion and a substantially straight second portion angularly disposed from the first portion by about 20 degrees.

14. The apparatus of claim 13, wherein the handle further includes a substantially straight third portion extending between the first portion and the second portion, the third portion extends from the first portion at an angle of about 90 degrees, and the second portion extends from the third portion at an angle of about 70 degrees.

15. The apparatus of claim 12, wherein the tibial sizing plate includes a substantially planar surface and defines a peripherally positioned pin slot extending obliquely relative to the substantially planar surface.

16. The apparatus of claim 15, wherein the tibial sizing plate is configured such that when the substantially planar surface faces inferiorly the pin slot is additionally positioned generally anteriorly of the substantially planar surface.

17. The apparatus of claim 16, wherein the tibial sizing plate further includes a flared portion, at least a portion of the pin slot extends through the flared portion, and the handle is coupled to the flared portion.

18. The apparatus of claim 16, wherein the tibial sizing plate further includes a notched portion, at least a portion of the pin slot extends through the notched portion, and the handle is coupled to the notched portion.

19. The apparatus of claim 16, further comprising:
a catch member coupled to the tibial sizing plate;
wherein the catch member is positioned generally posteriorly of the substantially planar surface, and the catch member is movable between a first position in which the catch member extends generally inferiorly from the tibial sizing plate and a second position in which the catch member retracts generally superiorly into the tibial sizing plate.

20. The apparatus of claim 19, wherein the catch member is coupled to a peripheral portion of the tibial sizing plate.

21. The apparatus of claim 20, wherein the catch member includes a pin.

22. The apparatus of claim 21, wherein the pin includes an annular notch.

23. A tibial sizing apparatus, comprising:
a tibial sizing plate; and
a first catch member coupled to a first peripheral portion of the tibial sizing plate;
wherein the first catch member is movable between a first first catch member position in which the first catch member extends generally inferiorly from the tibial sizing plate and a second first catch member position in which the first catch member retracts generally superiorly into the tibial sizing plate.

24. The apparatus of claim 23, wherein the tibial sizing plate includes titanium.

25. The apparatus of claim 23, further comprising:
a second catch member coupled to a second peripheral portion of the tibial sizing plate;
wherein the second catch member is movable, independently of the first catch member, between a first second catch member position in which the second catch member extends generally inferiorly from the tibial sizing plate and a second second catch member position in which the second catch member retracts generally superiorly into the tibial sizing plate.

26. The apparatus of claim 25, wherein:
the tibial sizing plate includes a substantially planar surface,
the first catch member is positioned generally posteriorly of the substantially planar surface and generally medially of the second catch member, and
the second catch member is positioned generally posteriorly of the substantially planar surface and generally laterally of the first catch member.

27. An apparatus for fixing a tibial sizing plate to a proximal tibia, the apparatus comprising:
means for anteriorly manipulating the tibial sizing plate;
means for posteriorly aligning the tibial sizing plate; and
means for anteriorly pinning the tibial sizing plate to the proximal tibia.

28. A method for fixing a tibial sizing plate to a proximal tibia, the method comprising the steps of:
anteriorly pinning the tibial sizing plate to the proximal tibia.

29. The method of claim 28, further comprising the steps of:
anteriorly manipulating the tibial sizing plate; and
posteriorly aligning the tibial sizing plate.

30. The method of claim 28, further comprising the step of:
positioning a tibio-femoral insert on the tibial sizing plate;
wherein the pinning step is executed while the tibio-femoral insert is positioned on the tibial sizing plate.
